(19) United States
(12) Patent Application Publication
(10) Pub. No.: US 2013/0184729 A1
(43) Pub. Date: Jul. 18, 2013

(54) ULTRASONIC THERAPEUTIC APPARATUS

(71) Applicant: OLYMPUS CORPORATION, Tokyo (JP)

(72) Inventor: Shinji YASUNAGA, Tokyo (JP)

(73) Assignee: OLYMPUS CORPORATION, Tokyo (JP)

(21) Appl. No.: 13/788,618

(22) Filed: Mar. 7, 2013

Related U.S. Application Data
(63) Continuation of application No. PCT/JP2011/074901, filed on Oct. 28, 2011.

(30) Foreign Application Priority Data
Nov. 8, 2010 (JP) 2010-249508

(51) Int. Cl. A61N 7/00

(52) U.S. Cl. CPC A61N 7/00
USPC 606/169

(57) ABSTRACT

There is provided an ultrasonic therapeutic apparatus, which is capable of shortening a surgery time even in a case of resecting and stopping bleeding of a plurality of sites.

The ultrasonic therapeutic apparatus includes a probe which is interpolated into a sheath, and a treatment section which is connected to or formed integrally with a front-end portion of the probe, and which his exposed to an exterior of the sheath, and a diseased part is resected or coagulated by making propagate ultrasonic waves to the treatment portion via the probe, and the ultrasonic therapeutic apparatus further includes a mechanism which cools down the treatment portion by carrying out heat exchange by the probe and a coolant gas which has been discharged into the sheath.

### TECHNICAL FIELD

The present invention relates to an ultrasonic therapeutic apparatus.

### BACKGROUND ART

FIG. 6 shows an overall arrangement of an ultrasonic cutting and coagulating system 10 which has hitherto been used. In the ultrasonic cutting and coagulating system 10, vibration of an ultrasonic transducer, upon being amplified by a horn, is transmitted to a fixed blade 34 which formed at a front end of a probe, via the probe (vibration transmission rod) connected to the horn. A movable blade 42 is pivotably retractable with respect to the fixed blade 34 via an operating rod. The probe and the operating rod are accommodated in a sheath 55, and are supported inside the sheath 55 by supporting members which are disposed at a predetermined distance inside the sheath 55. It is possible to resect while cauterizing a tissue sandwiched between the fixed blade 34 and the movable 42 by frictional heat due to ultrasonic waves, and also possible to stop bleeding simultaneously.

### CITATION LIST

### Patent Literature

Patent Document 1: Japanese Patent Application Laid-open Publication No. 2002-186627

### SUMMARY OF INVENTION

### Technical Problem

Generally, in one surgery, resection or coagulation of a plurality of locations becomes necessary. Here, in a structure described in Patent Document 1, after the treatment, the fixed blade 34 and a surrounding site thereof are at a high temperature. Therefore, there is a possibility of an injury caused due to a high-temperature part of a treatment tool making a contact with a site other than a diseased part. For preventing an injury caused due to such high-temperature site, it is necessary to wait till the temperature of the fixed blade 34 comes down, and it is not possible to carry out the subsequent treatment. Therefore, there has been a problem of a time for surgery becoming long.

Accordingly, installing of a cooling mechanism which has a small-size and an improved performance, to be incorporated in the treatment tool has been sought. During the treatment, the fixed blade is required to attain high temperature in a short time for exerting an effect of resecting and stopping bleeding. The cooling mechanism is sought to be being capable of cooling down the fixed blade 34 in short time after the end of treatment, with a quick response, without hindering the rise in temperature of the fixed blade by ultrasonic waves, by stopping functioning during the treatment.

The present invention has been made in view of the abovementioned issues, and an object of the present invention is to provide an ultrasonic therapeutic apparatus which is capable of shortening a surgery time even in a case of resecting and stopping bleeding of the plurality of parts.

### Solution to Problem

To solve the abovementioned issues and to achieve the object, an ultrasonic therapeutic apparatus according to the present invention includes

a probe which is interpolated into a sheath, and

a treatment section which is connected to or formed integrally with a front-end portion of the probe, and which is exposed to an exterior of the sheath, and

a diseased part is resected or coagulated by making propagate ultrasonic waves to the treatment section via the probe, and

the ultrasonic therapeutic apparatus further includes,

a mechanism which cools down the treatment section by carrying out heat exchange by the probe and a coolant gas which has been discharged into the sheath.

Moreover, according to a preferable aspect of the present invention, it is desirable that the coolant gas is supplied by infusing a high-pressure gas through an infusion tube having a thin diameter which is disposed inside the sheath, and furthermore, by cooling down the high-pressure gas at a front-end portion of the infusion tube.

Furthermore, according to a preferable aspect of the present invention, it is desirable that the infusion tube is interpolated into a reflux tube which is disposed inside the sheath, and the coolant gas which has been discharged from the infusion tube flows into the reflux tube and is discharged to the exterior of the sheath, and heat exchange is carried out between the high-pressure gas which is infused into the infusion tube and the coolant gas which is discharged from the reflux tube.

According to a preferable aspect of the present invention, it is desirable that the infusion tube is detachable from the reflux tube.

Moreover, according to a preferable aspect of the present invention, it is desirable that the reflux tube is detachable from the sheath.

Furthermore, according to a preferable aspect of the present invention, it is desirable that the infusion tube includes an infusion portion through which the high-pressure gas is to be infused, and a depressurizing portion having a diameter thinner than the diameter of the infusion portion which is formed at the front-end portion.

### Advantageous Effects of Invention

The ultrasonic therapeutic apparatus according to the present invention has a Joule-Thomson cooler having a thin double-tube structure incorporated inside a sheath which forms a treatment tool. A front end of a probe is to be cooled down from inside of the sheath, and it is possible to cool down by heat transfer by conduction between the front end of the probe and the treatment tool. Accordingly, the ultrasonic therapeutic apparatus according to the present invention shows an effect that it is possible to shorten a surgery time even in a case of resecting and stopping bleeding of a plurality of sites.

### BRIEF DESCRIPTION OF DRAWINGS

FIG. 1 is a diagram showing a structure of a treatment tool according to an embodiment of the present invention;

FIG. 2 is a diagram showing a structure of a JT cooler in the embodiment;

FIG. 3 is a diagram showing a state in which the JT cooler in the embodiment is divided into a reflux tube and an infusion tube;

FIG. 4 is a diagram for explaining an operation of resecting, stopping bleeding, and cooling down a diseased part by letting ultrasonic waves to be propagated to a treatment section in the embodiment;

FIG. 5 is an explanatory diagram of an operation of the JT cooler in the embodiment; and

FIG. 6 is a diagram showing an overall arrangement of a conventional ultrasonic cutting and coagulating system.

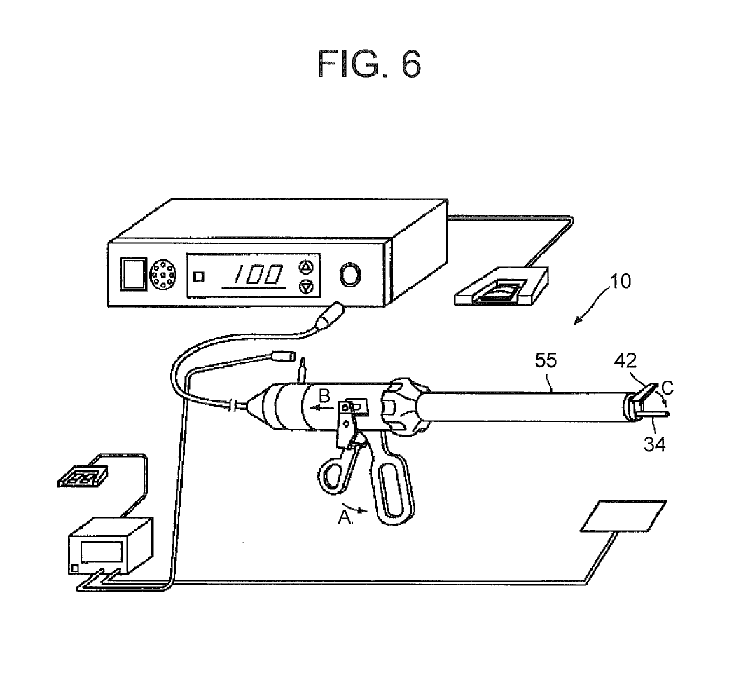

### DESCRIPTION OF EMBODIMENTS

An embodiment of an ultrasonic therapeutic apparatus according to the present invention will be described below in detail by referring to the accompanying diagrams. However, the present invention is not restricted to the embodiment described below.

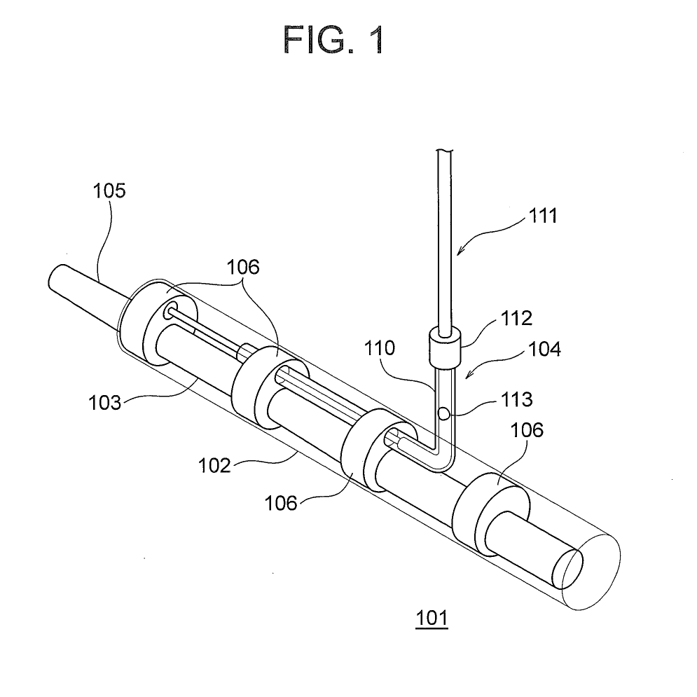

The ultrasonic therapeutic apparatus according to the present invention will be described by referring to diagrams from FIG. 1 to FIG. 5. FIG. 1 is a diagram showing a treatment tool of the ultrasonic therapeutic apparatus according to the present invention. Here, for simplifying the description, a characteristic structure of the embodiment is described below. Practically, the structure also includes a movable blade and a movable mechanism and a transducer which is connected to a rear end of a probe (not shown in the diagram).

A treatment tool 101 has a structure in which, a probe 103 is interpolated into a sheath 102, and a JT (Joule-Thomson) cooler 104 for cooling down the probe 103 is incorporated into a gap between the sheath 102 and the probe 103. A positional relationship of the sheath 102, the probe 103, and the JT cooler 104 is regulated by supporting members 106. Moreover, a treatment portion 105 which is formed integrally with a front end of the probe 103 is protruded from the sheath 102 and exposed to an exterior. A structure of the JT cooler 104 (will be described later) is a structure in which, a part thereof is protruded from the sheath 102 as shown in FIG. 1. An interior of the sheath 102 is divided into a plurality of sites by the supporting members 106, and the sites of the sheath 102 have a mutually air-tight structure to certain extent.

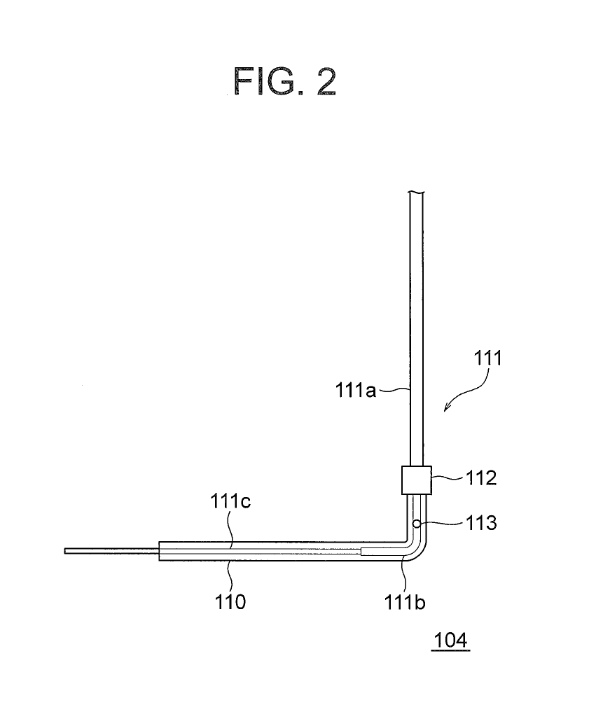

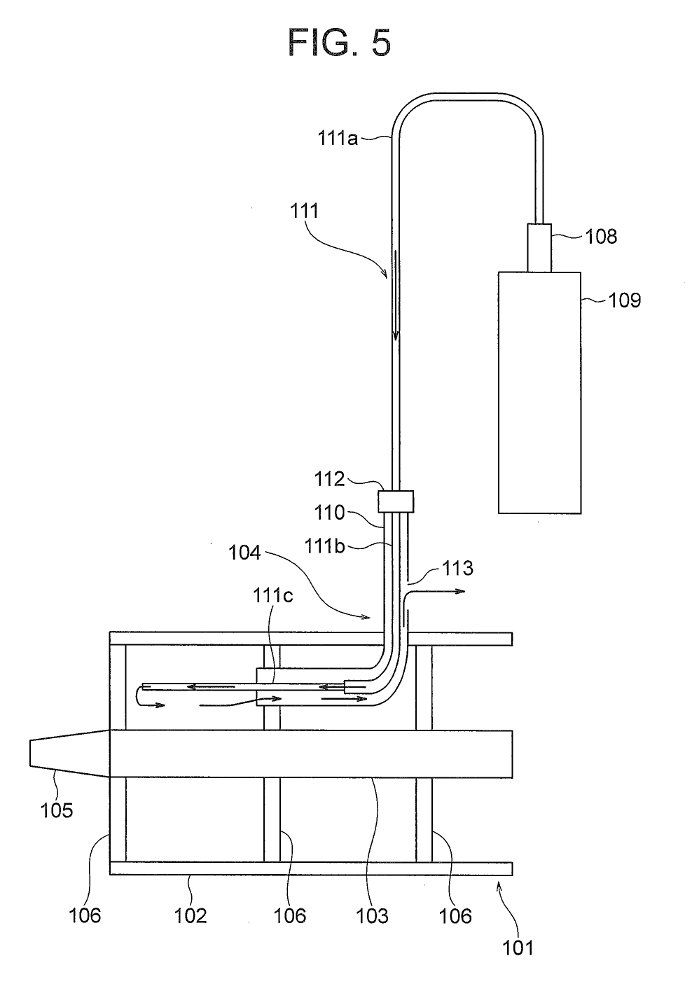

FIG. 2 shows a structure of the JT cooler 104. The JT cooler 104 has a structure in which, an infusion tube 111 is interpolated into a reflux tube 110. The infusion tube 111 is connected to a gas cylinder (will be described later, refer to FIG. 5). The infusion tube 111 is formed of three portions namely, a rear-end portion of the reflux tube 110, or in other words, a rear-end side infusion portion 111a which is protruded from a portion toward an operator, an infusion-tube front-end side infusion portion 111b which is to be interpolated into the reflux tube 110, and a front-end side of the front-end side infusion portion 111b, or in other words, a depressurizing portion 111c which is fitted on a side of the treatment tool.

Moreover, an inner diameter of the depressurizing portion 111c is smaller than an inner diameter of the rear-end side infusion portion 111a and an inner diameter of a front-end side infusion portion 111b. The reflux tube 110 and the infusion tube 111 are connected by a tube connector 112 and are detachable. Moreover, a gas discharge hole 113 is formed at a site near the tube connector 112 of the reflux tube 110.

Both a front end with the reflux tube 110 and a front end of the depressurizing portion 111c of the infusion tube are open. From among the plurality of sites inside the sheath 102 shown in FIG. 1 which are partitioned by the supporting members 106, both are positioned at a front-end side of the probe 103, or in other words, positioned near the treatment portion side.

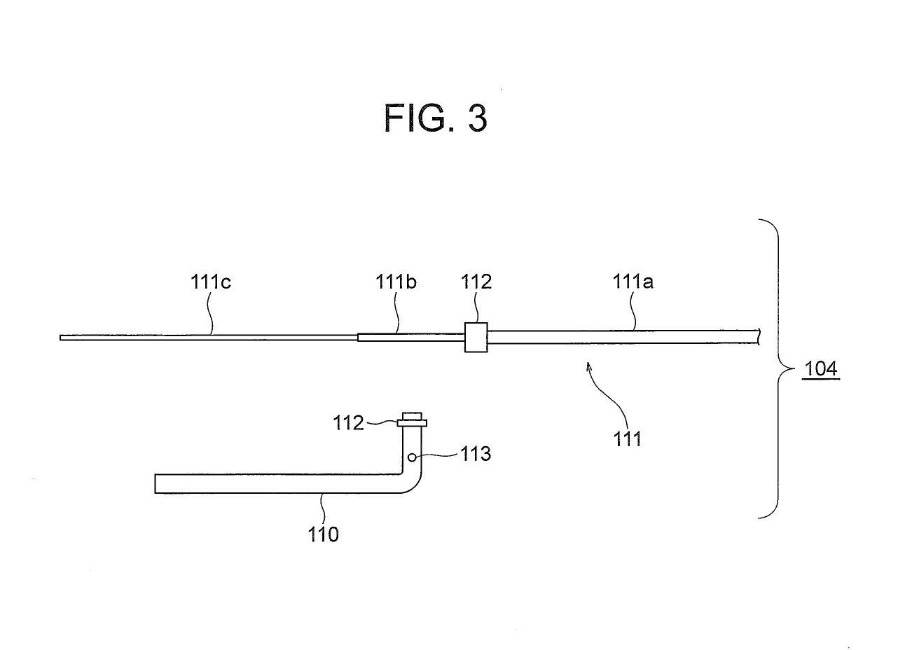

FIG. 3 shows a state in which the JT cooler 104 is divided into the reflux tube 110 and the infusion tube 111. The infusion tube 111 is flexible, and can be inserted into the reflux tube 110, and connected by the tube connector 112.

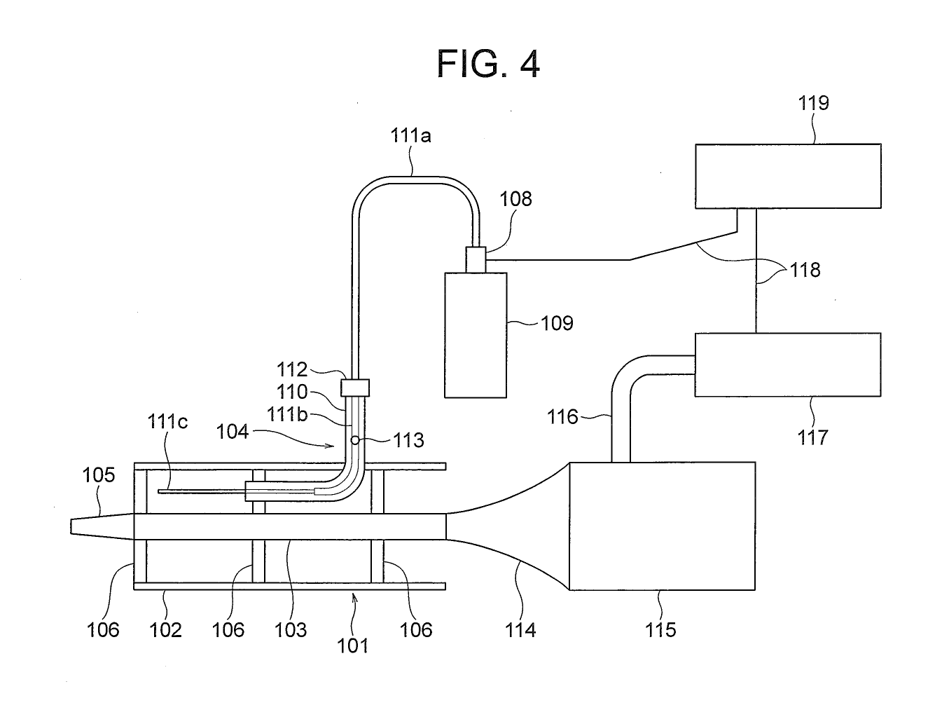

FIG. 4 is a diagram showing a schematic structure for explaining an operation of resecting, stopping bleeding, and cooling down a diseased part by letting ultrasonic waves to be propagated to the treatment portion 105. Ultrasonic vibration which is generated in a BLT 115 (Bolt-clamped Langevin-type Transducer) is amplified by passing through a horn 114, and makes vibrate the probe 103 and the treatment portion 105. The JT cooler 104 cools down the probe 103 and the front-end portion by discharging CO₂ gas. An operation of the JT cooler 104 will be described later.

Treatment of resecting and stopping bleeding is carried out by vibration of the treatment portion 105. Treatment of resecting and stopping bleeding of the diseased part and cooling down of the probe 103 are controlled by opening and closing of a solenoid valve 108 of a CO₂ gas cylinder 109 and a driving power source 117 of the BLT 115. The abovementioned control is carried out by a system controller 119. After the operator has completed the treatment, by the control of the system controller 119, it is possible to realize lowering of temperature of the probe 103 and eventually lowering of temperature of the treatment portion 105 in a short time by opening the solenoid valve 108 of the CO₂ gas cylinder 109 for a predetermined amount of time.

FIG. 5 is a diagram showing a schematic structure for explaining the operation of the JT cooler 104. The high-pressure CO₂ gas which has been infused into the infusion tube 111 causes a sudden pressure drop by passing through the depressurizing portion 111c having a small inner diameter. Accordingly, a drop in temperature of the gas occurs due to Joule-Thomson effect. The CO₂ gas which has been cooled down is discharged from the front end of the depressurizing portion 111c and carries out heat exchange with the probe 103, and lowers the temperature of the probe 103.

Furthermore, the CO₂ gas which has been cooled down is discharged from the front end of the depressurizing portion 111c and flows into the reflux tube 110. At this time, by lowering the temperature of the probe 103, the temperature of the treatment portion 105 is also lowered due to heat transfer by conduction. Here, it is preferable to set the inner diameter and a length of the depressurizing portion 111c such that a pressure of the gas in a state of being discharged from the depressurizing portion 111c becomes slightly higher than 1 atm. Accordingly, it is possible to let the temperature drop due to the Joule-Thomson effect to be the maximum. At the same time, since the purpose is served even when a pressure resistance of the sheath 102 which has been partitioned by the supporting member 106 is not high, it is advantageous from a point of small-sizing and cost.

The cooled-down CO₂ gas which has flowed into the reflux tube 110 retains a low temperature to certain extent. The cooled-down CO₂ gas carries out heat exchange with the CO₂ gas which is infused into the front-end side infusion portion 111b of the infusion tube 111 inside the reflux tube 110. Accordingly, the CO₂ gas which is infused is cooled down at a point of time of reaching the depressurizing portion 111c. In other words, for the CO₂ gas which is discharged from the depressurizing portion 111c, in addition to the temperature drop due to the Joule-Thomson effect in the depressurizing portion 111c, it is possible to achieve an effect of further temperature drop. Therefore, it is possible to lower the temperature of the CO₂ gas which is discharged, close to a liquefaction temperature of the CO₂ gas by optimizing the design. The CO₂ gas which flows into the reflux tube 110 is eventually discharged through a gas discharge hole 113 in the reflux tube 110 provided at an exterior of the sheath 102.

In FIG. 5, the gas discharge hole 113 is drawn to be open toward a rightward horizontal direction with respect to a paper surface so that a route shown by arrows for gas discharge is easily understandable. The gas discharge hole 113, without being restricted to the abovementioned type, can also be let to open in a direction toward a front surface with respect to the paper surface as shown in FIG. 2, FIG. 3, and FIG. 4.

In the embodiment described above, the Joule-Thomson cooler having a thin double-tube structure is incorporated inside the sheath which forms the treatment tool. The front end of the probe is to be cooled down from inside of the sheath, and it is possible to cool down by heat transfer by conduction between the front end of the probe and the treatment tool exposed to an exterior of the sheath which is connected to or formed integrally with the front-end portion of the probe. Accordingly, an effect is shown that the temperature of the front end of the probe by which, treatment of plurality of sites is carried out, is lowered in a short time, and also, even by stopping the cooler during the treatment, there is almost no increase in the treatment time.

The present invention may have various modified embodiments without departing from the scope of the invention.

### INDUSTRIAL APPLICABILITY

As it has been described above, the ultrasonic therapeutic apparatus according to the present invention being capable of cooling a treatment portion in a short time is useful for carrying out a treatment of resecting and stopping bleeding of the plurality of sites.

### REFERENCE SIGNS LIST

101 treatment tool
102 sheath
103 probe
104 JT cooler
105 treatment portion
106 supporting member
108 solenoid valve
109 CO₂ cylinder
110 reflux tube
111 infusion tube
111a rear-end side infusion portion of infusion tube
111b front-end side infusion portion of infusion tube
111c depressurizing portion of infusion tube
112 tube connector
113 gas discharge hole
114 horn
115 BLT (Bolt-clamped Langevin-type Transducer)
116 drive-voltage supply cable
117 driving power source
118 control signal cable
119 system controller

1. An ultrasonic therapeutic apparatus comprising:
a probe which is interpolated into a sheath; and
a treatment portion which is connected to or formed integrally with a front-end portion of the probe, and which is exposed to an exterior of the sheath,
wherein a diseased part is resected or coagulated by making propagate ultrasonic waves to the treatment portion via the probe, and
further comprising:
a mechanism which cools down the treatment portion by carrying out heat exchange by the probe and a coolant gas which has been discharged into the sheath.

2. The ultrasonic therapeutic apparatus according to claim 1, wherein the coolant gas is supplied by infusing a high-pressure gas through an infusion tube having a thin diameter which is disposed inside the sheath, and furthermore, by cooling down the high-pressure gas at a front-end portion of the infusion tube.

3. The ultrasonic therapeutic apparatus according to claim 2, wherein the infusion tube is interpolated into a reflux tube which is disposed inside the sheath, and the coolant gas which has been discharged from the infusion tube flows into the reflux tube and is discharged to the exterior of the sheath, and heat exchange is carried out between the high-pressure gas which is infused into the infusion tube and the coolant gas which is discharged from the reflux tube.

4. The ultrasonic therapeutic apparatus according to claim 3, wherein the infusion tube is detachable from the reflux tube.

5. The ultrasonic therapeutic apparatus according to claim 3, wherein the reflux tube is detachable from the sheath.

6. The ultrasonic therapeutic apparatus according to claim 2, wherein the infusion tube includes an infusion portion through which the high-pressure gas is to be infused, and a depressurizing portion having a diameter thinner than the diameter of the infusion portion which is formed at the front-end portion.
